(19) United States
(12) Patent Application Publication
(10) Pub. No.: US 2001/0031891 A1
(43) Pub. Date: Oct. 18, 2001

(54) Process for producing perfluoroalkanesulfinate

(76) Inventors: Yoshihiko Goto, Saitama (JP); Shigenori Sakai, Yamaguchi (JP); Hiroaki Sakaguchi, Yamaguchi (JP)

Correspondence Address: CROWELL & MORING LLP, Intellectual Property Group, P.O. Box 14300, Washington, DC, 20044-4300, US

(21) Appl. No.: 09/788,655

(22) Filed: Feb. 21, 2001

(30) Foreign Application Priority Data
Feb. 22, 2000 (JP) 2000-043870
Feb. 13, 2001 (JP) 2001-036032

(51) Int. Cl. C 07C 3 9/04

(52) U.S. Cl. 562/119

(57) ABSTRACT

The invention relates to a process for producing an alkali metal salt of a perfluoroalkanesulfinic acid. This process includes the step of bringing a perfluoroalkanesulfonic fluoride into contact with an alkali metal salt of sulfurous acid in the presence of water. This process is useful, since this perfluoroalkanesulfonic fluoride (e.g., trifluoromethanesulfonic fluoride) is easily available.

### BACKGROUND OF THE INVENTION

The present invention relates to a process for producing an alkali metal salt of a perfluoroalkanesulfinic acid, which is an important compound as an intermediate for medicines and agricultural chemicals.

R. M. Scribner, J. Org. Chem., 31, 3671 (1966) discloses that potassium trifluoromethanesulfinate is obtained by reacting trifluoromethanesulfonyl chloride with potassium sulfite. Japanese Patent Laid-open Publication JP-A-48-56619 discloses that trifluoromethanesulfinic acid is obtained by reacting trifluoromethanesulfonic fluoride with hydrazine. H. W. Roesky and G. Holtscheider, J. Fluorine Chemistry, 7,77 (1976) discloses that trifluoromethanesulfinic acid reacts with sodium carbonate or potassium carbonate, thereby obtaining a corresponding salt. Trifluoromethanesulfonyl chloride is a relatively expensive raw material for producing an alkali metal salt of trifluoromethanesulfinic acid, since it is common to produce trifluoromethanesulfonyl chloride from trifluoromethanesulfonyl fluoride.

### SUMMARY OF THE INVENTION

It is therefore an object of the present invention to provide a process for producing an alkali metal salt of a perfluoroalkanesulfinic acid using a raw material that is easily available in an industrial scale.

According to the present invention, there is provided a process for producing a first alkali metal salt of a perfluoroalkanesulfinic acid. This process comprises bringing a perfluoroalkanesulfonic fluoride into contact with a second alkali metal salt of sulfurous acid in the presence of water.

### DESCRIPTION OF THE PREFERRED EMBODIMENTS

The process can be conducted, for example, by bringing trifluoromethanesulfonic fluoride into contact with a sodium sulfite or potassium sulfite aqueous solution, thereby producing the first alkali metal salt, that is, sodium trifluoromethanesulfinate or potassium trifluoromethanesulfinate.

A perfluoroalkanesulfonic fluoride can be obtained by electrolytic fluorination of a sulfonic fluoride (having a carbon atom number corresponding to that of the perfluoroalkanesulfonic fluoride) using a potassium fluoride fused salt. Examples of a perfluoroalkanesulfonic fluoride used in the process are those having a carbon atom number of about 1-20, such as trifluoromethanesulfonic fluoride, pentafluoroethanesulfonic fluoride, heptafluoropropanesulfonic fluoride, nonafluorobutanesulfonic fluoride, undecafluoropentanesulfonic fluoride, tridecafluorohexanesulfonic fluoride, pentadecafluoroheptanesulfonic fluoride, and isomers of these. Among them, those having a carbon atom number of about 1-8 are preferable. Of these examples, trifluoromethanesulfonic fluoride, pentafluoroethanesulfonic fluoride, heptafluoropropanesulfonic fluoride, and nonafluorobutanesulfonic fluoride are preferable.

The second alkali metal salt (i.e., an alkali metal salt of sulfurous acid) can be a salt of an alkali metal selected from lithium, sodium, potassium, rubidium and cesium. Of these, sodium and potassium are preferable. The amount of the second alkali metal salt can be 1 mol or greater, preferably not greater than about 10 moles, more preferably not greater than about 4 moles, per mol of the perfluoroalkanesulfonic fluoride.

During the reaction, it is preferable to adjust the reaction system to having a pH of about 7-9 by properly adding a basic compound to the reaction system, since its pH tends to lower as the reaction proceeds. With this pH adjustment, the amount of the second alkali metal salt can be made to be as small as about 1-2 moles per mol of the perfluoroalkanesulfonic fluoride. Examples of the basic compound are hydroxides, oxides and carbonates of lithium, sodium, potassium, rubidium, and cesium. It is preferable that the metal of this basic compound is the same as that of the second alkali metal salt. Furthermore, it is preferable to add the basic compound in the form of an aqueous solution.

The amount of water used in the process can arbitrarily be selected. Its amount is preferably such that an aqueous solution obtained by dissolving the second alkali metal salt in water contains about 5-50 wt % of the second alkali metal salt. If the amount of water is too small, stirring for preparing this aqueous solution may become difficult. If it is too large, conducting a treatment after the reaction may become cumbersome. It is optional to add an organic solvent to the aqueous solution, such as a water-soluble organic solvent (e.g., acetone, acetonitrile, THF and dioxane).

In the process, the reaction can be conducted at a temperature of 0-100° C., preferably 0-80° C. Since trifluoromethanesulfonic fluoride has a boiling point of −23° C., it is preferable to conduct the reaction under a pressurized condition of about 0-10 MPa using a pressure-proof reactor.

After the reaction, it is possible to conduct the following post-treatments to obtain the first alkali metal salt (i.e., an alkali metal salt of a perfluoroalkanesulfinic acid). At first, water is removed from the reaction liquid to precipitate a solid matter. Then, a solvent (e.g., acetone, acetonitrile, ethyl acetate, methanol and ethanol) that is capable of dissolving the first alkali metal salt is added to the solid matter. Then, a solid matter remaining in the solution is removed, followed by distilling the solvent out, thereby obtaining the first alkali metal salt.

The following nonlimitative examples are illustrative of the present invention.

### EXAMPLE 1

At first, an aqueous solution was prepared by dissolving 7.5 g of sodium sulfite in 28 g of water. Then, a metal pressure-proof reactor was charged with this aqueous solution. Then, the atmosphere of the reactor was removed to obtain a reduced pressure. After that, 8.5 g of trifluoromethanesulfonic fluoride were added to the reactor, followed by stirring at a temperature of from 0° C. to room temperature for 12 hr. The resulting reaction liquid was neutralized with sodium carbonate, followed by removal of water. Then, acetone was added to the remaining solid matter to extract the target product. The resulting acetone solution was concentrated to dryness, thereby obtaining 3.4 g of sodium trifluoromethanesulfinate (yield: 39%).

### EXAMPLE 2

At first, an aqueous solution was prepared by dissolving 5.5 g of potassium sulfite in 15 g of water. Then, a metal pressure-proof reactor was charged with this aqueous solution. Then, the atmosphere of the reactor was removed to obtain a reduced pressure. After that, 5.2 g of trifluoromethanesulfonic fluoride were added to the reactor, followed by stirring at a temperature of from 0° C. to room temperature for 12 hr. The resulting reaction liquid was neutralized with potassium carbonate, followed by removal of water. Then, acetone was added to the remaining solid matter to extract the target product. The resulting acetone solution was concentrated to dryness, thereby obtaining 2.8 g of potassium trifluoromethanesulfinate (yield: 47%).

### EXAMPLE 3

At first, an aqueous solution was prepared by dissolving 22 g of sodium sulfite in 90 g of water. Then, a metal pressure-proof reactor was charged with this aqueous solution. Then, the atmosphere of the reactor was removed to obtain a reduced pressure. After that, 11.6 g of pentafluoroethanesulfonic fluoride were added to the reactor, followed by stirring at a temperature of from 0° C. to room temperature for 48 hr. The resulting reaction liquid was neutralized with sodium carbonate, followed by removal of water. Then, methanol was added to the remaining solid matter to extract the target product. The resulting methanol solution was concentrated to dryness, thereby obtaining 10.7 g of sodium pentafluoroethanesulfinate (yield: 90%).

### EXAMPLE 4

At first, an aqueous solution was prepared by dissolving 18 g of sodium sulfite in 70 g of water. Then, a metal pressure-proof reactor was charged with this aqueous solution. Then, the atmosphere of the reactor was removed to obtain a reduced pressure. After that, 10.8 g of nonafluorobutanesulfonic fluoride were added to the reactor, followed by stirring at a temperature of from 0° C. to room temperature for 100 hr. The resulting reaction liquid was neutralized with sodium carbonate, followed by removal of water. Then, methanol was added to the remaining solid matter to extract the target product. The resulting methanol solution was concentrated to dryness, thereby obtaining 9.2 g of sodium nonafluorobutanesulfinate (yield: 88%).

### EXAMPLE 5

At first, an aqueous solution was prepared by dissolving 37.5 g of sodium sulfite in 150 g of water. Then, a metal pressure-proof reactor was charged with this aqueous solution. Then, the atmosphere of the reactor was removed to obtain a reduced pressure. After that, 10.7 g of trifluoromethanesulfonic fluoride were added to the reactor, followed by stirring at 5° C. for 4 hr. The resulting reaction liquid was neutralized with sodium carbonate, followed by removal of water. Then, methanol was added to the remaining solid matter to extract the target product. The resulting methanol solution was concentrated to dryness, thereby obtaining 9.7 g of sodium trifluoromethanesulfinate (yield: 88%).

### EXAMPLE 6

At first, an aqueous solution was prepared by dissolving 125 g of potassium sulfite in 290 g of water. Then, a metal pressure-proof reactor was charged with this aqueous solution. Then, the atmosphere of the reactor was removed to obtain a reduced pressure. After that, 30.7 g of trifluoromethanesulfonic fluoride were added to the reactor, followed by stirring at a temperature of from 0° C. to room temperature for 12 hr. The resulting reaction liquid was neutralized with potassium carbonate, followed by removal of water. Then, methanol was added to the remaining solid matter to extract the target product. The resulting methanol solution was concentrated to dryness, thereby obtaining 32.6 g of potassium trifluoromethanesulfinate (yield: 94%).

### EXAMPLE 7

At first, an aqueous solution was prepared by dissolving 36.3 g of sodium sulfite in 145 g of water. Then, a metal pressure-proof reactor equipped with a pH meter was charged with this aqueous solution. Then, the atmosphere of the reactor was removed to obtain a reduced pressure. After that, 30.7 g of trifluoromethanesulfonic fluoride were added to the reactor. The reaction was continued for 6 hr, while pH of the reaction system was maintained within a range of 7-9 by properly adding 20% NaOH aqueous solution using a pressure pump. This NaOH aqueous solution was added in a total amount of 115 g. After the reaction, the reaction liquid was taken out of the reactor, followed by removal of water. Then, acetone was added to the remaining solid matter to extract the target product. The resulting acetone solution was concentrated to dryness, thereby obtaining 33.7 g of sodium trifluoromethanesulfinate (yield: 75%).

The entire disclosure of Japanese Patent Application No. 2000-043870 filed on Feb. 22, 2000, including specification, claims and summary, is incorporated herein by reference in its entirety.

What is claimed is:

1. A process for producing a first alkali metal salt of a perfluoroalkanesulfinic acid, comprising bringing a perfluoroalkanesulfonic fluoride into contact with a second alkali metal salt of sulfurous acid in the presence of water.

2. A process according to claim 1, wherein an alkali metal contained in each of said first and second alkali metal salts is sodium or potassium.

3. A process according to claim 1, wherein said perfluoroalkanesulfonic fluoride has a carbon atom number of 1-20.

4. A process according to claim 1, wherein said perfluoroalkanesulfonic fluoride is selected from the group consisting of trifluoromethanesulfonic fluoride, pentafluoroethanesulfonic fluoride, heptafluoropropanesulfonic fluoride, and nonafluorobutanesulfonic fluoride.

5. A process according to claim 1, wherein said second alkali metal salt is in an amount of 1 mol or greater per mol of said perfluoroalkanesulfonic fluoride.

6. A process according to claim 1, wherein said perfluoroalkanesulfonic fluoride is brought into contact with an aqueous solution comprising about 5-50 wt % of said second alkali metal salt.

7. A process according to claim 1, wherein said bringing is conducted under a pressurized condition.

8. A process according to claim 1, wherein said process further comprising:
removing said water from a reaction product obtained by said bringing, thereby precipitating a solid matter;
adding a solvent to said solid matter to extract said first alkali metal salt, thereby obtaining a solution; and
removing said solvent from said solution to obtain said first alkali metal salt.

9. A process according to claim 1, wherein a reaction system comprising said perfluoroalkanesulfonic fluoride, said second alkali metal salt and said water is adjusted to having a pH of 7-9 by adding a basic compound to said reaction system, during a reaction between said perfluoroalkanesulfonic fluoride and said second alkali metal salt.
